(19) United States
(12) Patent Application Publication
(10) Pub. No.: US 2012/0083675 A1
(43) Pub. Date: Apr. 5, 2012

(54) MEASURING AFFECTIVE DATA FOR WEB-ENABLED APPLICATIONS

(76) Inventors: Rana el Kaliouby, Newton, MA (US); Rosalind Wright Picard, Newtonville, MA (US); Richard Scott Sadowsky, Sturbridge, MA (US)

(21) Appl. No.: 13/249,317

(22) Filed: Sep. 30, 2011

Related U.S. Application Data
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/0205; G09B 19/00

(52) U.S. Cl. 600/301; 434/236

(57) ABSTRACT

Mental state information is collected as a person interacts with a rendering such as a website or video. The mental state information is collected through video capture or capture of sensor information. The sensor information can be of electrodermal activity, accelerometer readings, skin temperature, or other characteristics. The mental state information is uploaded to a server and aggregated with other people's information so that collective mental states are associated with the rendering. The aggregated mental state information is displayed through a visual representation such as an avatar.

### RELATED APPLICATIONS

This application claims the benefit of U.S. provisional patent applications “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. Each of the foregoing applications is hereby incorporated by reference in its entirety.

### FIELD OF INVENTION

This application relates generally to analysis of mental states and more particularly to measuring affective data for web-enabled applications.

### BACKGROUND

People spend a tremendous amount of time on the internet, much of that including the viewing and interacting with web pages. Website analytics have been performed analyzing the amount of time which a person spends on a web page and the path through the internet which the person has taken. This type of analysis has been used to evaluate the value and benefit of web pages and the respective styles of these pages.

The evaluation of mental states is key to understanding individuals and the way in which they react to the world around them. Mental states run a broad gamut from happiness to sadness, from contentedness to worry, from excitement to calmness, as well as numerous others. These mental states are experienced in response to everyday events such as frustration during a traffic jam, boredom while standing in line, impatience while waiting for a cup of coffee, and even as people interact with their computers and the internet. Individuals may become rather perceptive and empathetic based on evaluating and understanding others' mental states but automated evaluation of mental states is far more challenging. An empathetic person may perceive another's being anxious or joyful and respond accordingly. The ability and means by which one person perceives another's emotional state may be quite difficult to summarize and has often been communicated as having a “gut feel.”

Many mental states, such as confusion, concentration, and worry, may be identified to aid in the understanding of an individual or group of people. People can collectively respond with fear or anxiety, such as after witnessing a catastrophe. Likewise, people can collectively respond with happy enthusiasm, such as when their sports team obtains a victory. Certain facial expressions and head gestures may be used to identify a mental state that a person is experiencing. Limited automation has been performed in the evaluation of mental states based on facial expressions. Certain physiological conditions may provide telling indications of a person's state of mind and have been used in a crude fashion as in an apparatus used for lie detector or polygraph tests.

### SUMMARY

Analysis of people, as they interact with the internet, may be performed by gathering mental states through evaluation of facial expressions, head gestures, and physiological conditions. This analysis may be connected to specific interactions with web pages or portions of a given web page. A computer implemented method for analyzing web-enabled application traffic is disclosed comprising: collecting mental state data from a plurality of people as they interact with a rendering; uploading information, to a server, based on the mental state data from the plurality of people who interact with the rendering; receiving aggregated mental state information on the plurality of people who interact with the rendering; and displaying the aggregated mental state information with the rendering. The aggregated mental state information may include norms derived from the plurality of people. The norms may be based on contextual information. The method may further comprise associating the aggregated mental state information with the rendering. The method may further comprise inferring of mental states based on the mental state data collected from the plurality of people. The rendering may be one of a group comprising a button, an advertisement, a banner ad, a drop down menu, and a data element on a web-enabled application. The rendering may be one of a group comprising a landing page, a checkout page, a webpage, a website, a web-enabled application, a video on a web-enabled application, a game on a web-enabled application, and a virtual world. The collecting mental state data may involve capturing of one of a group comprising physiological data and facial data. A webcam may be used to capture one or more of the facial data and the physiological data. The physiological data may be used to determine autonomic activity. The autonomic activity may be one of a group comprising heart rate, respiration, and heart rate variability. The facial data may include information on one or more of a group comprising facial expressions, action units, head gestures, smiles, brow furrows, squints, lowered eyebrows, raised eyebrows, and attention. The method may further comprise tracking of eyes to identify the rendering with which interacting is accomplished. The tracking of the eyes may identify a portion of the rendering on which the eyes are focused. A webcam may be used to track the eyes. The method may further comprise recording of eye dwell time on the rendering and associating information on the eye dwell time to the rendering and to the mental states. The interacting may include one of a group comprising viewing, clicking, and mousing over. The method may further comprise opting in, by an individual from the plurality of people, to allowing facial information to be aggregated. The method may further comprise opting in, by an individual from the plurality of people, to allowing uploading of information to the server.

Aggregation of the aggregated mental state information may be accomplished using computational aggregation. In some embodiments, aggregation of the aggregated mental state information may be performed on a demographic basis so that mental state information is grouped based on the demographic basis. The method may further comprise creating a visual representation of one or more of the aggregated mental state information and mental state information on an individual from the plurality of people. The visual representation may display the aggregated mental state information on a demographic basis. The method may further comprise animating an avatar to represent one or more of the aggregated mental state information and mental state information on an individual from the plurality of people. The method may further comprise synchronizing the aggregated mental state information with the rendering. The method may further comprise capturing contextual information about the rendering. The contextual information may include one or more of a timeline, a progression of web pages, or an actigraph. The mental states may include one of a group comprising frustration, confusion, disappointment, hesitation, cognitive overload, focusing, being engaged, attending, boredom, exploration, confidence, trust, delight, and satisfaction.

In embodiments, a computer program product embodied in a non-transitory computer readable medium for analyzing web-enabled application traffic, may comprise: code for collecting mental state data from a plurality of people as they interact with a rendering; code for uploading information, to a server, based on the mental state data from the plurality of people who interact with the rendering; code for receiving aggregated mental state information on the plurality of people who interact with the rendering; and code for displaying the aggregated mental state information with the rendering. In embodiments, a system for analyzing web-enabled application traffic states may comprise: a memory which stores instructions; one or more processors attached to the memory wherein the one or more processors, when executing the instructions which are stored, are configured to: collect mental state data from a plurality of people as they interact with a rendering; upload information, to a server, based on the mental state data from the plurality of people who interact with the rendering; receive aggregated mental state information on the plurality of people who interact with the rendering; and display the aggregated mental state information with the rendering.

In some embodiments, a method for analyzing web-enabled application traffic may comprise: receiving mental state data collected from a plurality of people as they interact with a rendering, receiving aggregated mental state information on the plurality of people who interact with the rendering; and displaying the aggregated mental state information with the rendering. In some embodiments, a computer implemented method for analyzing web-enabled application traffic may comprise: receiving mental state data collected from a plurality of people as they interact with a rendering, aggregating mental state information on the plurality of people who interact with the rendering; associating the aggregated mental state information with the rendering; and providing the aggregated mental state information to a requester. In embodiments, a computer implemented method for analyzing renderings on electronic displays may comprise: interacting with a rendering on an electronic display by a first person; capturing data on the first person into a computer system as the first person interacts with the rendering on the electronic display; inferring of mental states for the first person who interacted with the rendering based on the data which was captured for the first person; uploading information to a server on the data which was captured on the first person; interacting with the rendering by a second person; capturing data on the second person as the second person interacts with the rendering; inferring of mental states for the second person who interacted with the rendering based on the data which was captured for the second person; uploading information to the server on the data which was captured on the second person; aggregating information on the mental states of the first person with the mental states of the second person resulting in aggregated mental state information; and associating the aggregated mental state information to the rendering with which the first person and the second person interacted.

Various features, aspects, and advantages of numerous embodiments will become more apparent from the following description.

### BRIEF DESCRIPTION OF THE DRAWINGS

The following detailed description of certain embodiments may be understood by reference to the following figures wherein:

FIG. 1 is a flowchart for providing affect analysis for multiple people.

FIG. 2 is a diagram representing physiological analysis.

FIG. 3 is a diagram of heart related sensing.

FIG. 4 is a diagram of for capturing facial response to rendering.

FIG. 5 is a flowchart for performing facial analysis.

FIG. 6 is a flowchart for using mental state information.

FIG. 7 is a flowchart for opting into analysis.

FIG. 8 is a representative diagram of a rendering and response.

FIG. 9 is a representative diagram of a rendering and an aggregated response.

FIG. 10 is a representative diagram of a rendering and response with avatar.

FIG. 11 is a graphical representation of mental state analysis.

FIG. 12 is a graphical representation of mental state analysis along with an aggregated result from a group of people.

FIG. 13 is a flowchart for analyzing affect from rendering interaction.

FIG. 14 is an example embodiment of a visual representation of mental states.

FIG. 15 is a diagram of a system for analyzing web-enabled application traffic states utilizing multiple computers.

### DETAILED DESCRIPTION

The present disclosure provides a description of various methods and systems for analyzing people's mental states as they interact with websites and other features on the internet. A mental state may be an emotional state or a cognitive state. Examples of emotional states include happiness or sadness. Examples of cognitive states include concentration or confusion. Observing, capturing, and analyzing these mental states can yield significant information about people's reactions to websites that far exceed current capabilities in website analytics.

A challenge solved by this disclosure is the analysis of mental states within a web-oriented environment. Information on mental states may be collected on a client machine and either uploaded to a server raw or analyzed and abstracted, followed by uploading. The cloud based system may perform analysis on the mental states as an individual or group interact with videos, advertisements, web pages, and the like based on the mental state information which was uploaded. The mental state information may be aggregated across a group of people to provide summaries on people's mental states as they interact with web-enabled applications. The aggregated information may provide normative criteria that are important for comparing customer experiences across different applications and across common experiences within many applications such as online payment or point of sale. The applications may be web pages, web sites, web portals, mobile device applications, dedicated applications, and similar web-oriented tools and capabilities. The aggregated mental state information may be downloaded to the original client machine where the mental state information was uploaded from or alternately downloaded to another client machine for presentation. Mental states, which have been inferred based on the mental state information, may then be presented on a client machine display along with a rendering showing the material with which people interacted.

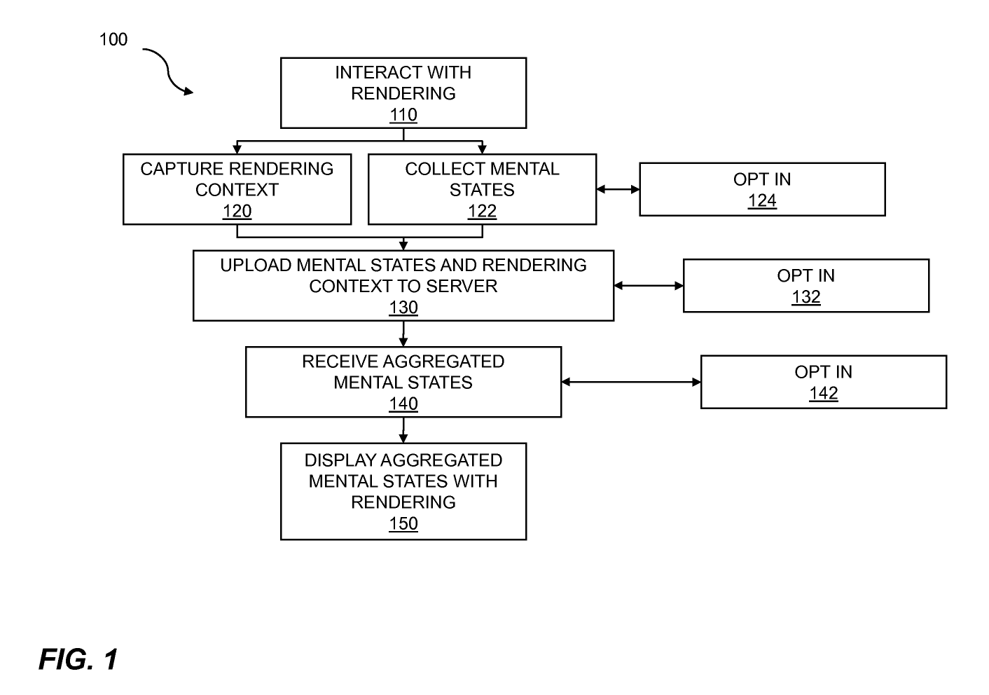

FIG. 1 is a flowchart for providing affect analysis for multiple people. The process may include a method for analyzing web-enabled application traffic. The flow 100 begins with a person or persons interacting with a rendering 110. The process may include interacting with a rendering by a plurality of people. A rendering may include a landing page, a checkout page, a webpage, a website, a web-enabled application, a video on a web-enabled application, a game on a web-enabled application, a virtual world, or other visible outputs of various web-enabled applications. A rendering may also include, among other items, a portion of one of items such as a button, an advertisement, a banner ad, a drop down menu, a section of text, an image, and a data element on a web-enabled application. The interacting with the rendering may include a variety of types of interaction, including viewing, clicking, typing, filling in form data, mousing over the rendering or any type of human-machine interaction. Flow 100 may continue with capturing contextual information about the rendering 120. The context may be any information related to the rendering, such as a timeline, a progression of web pages, an actigraph, demographic information about the individual interacting with the rendering, or any other type of information related to the rendering, the individual, or the circumstances of the interaction. The timeline may include information on when a rendering was interacted with or viewed. For instance, a video may be viewed and the times when mental states were collected along with the corresponding time points in the video may be recorded. In some embodiments, the contextual information may include a progression of web pages. A progression of web pages may include the uniform resource locators (URL) viewed and the order in which they are recorded. By collecting a progression of web pages, collected mental states may be correlated with the web pages viewed.

Flow 100 continues with collecting mental state data 122 from a plurality of people as they interact with a rendering. Mental state data that may be collected includes physiological data, facial data, other images, sounds, timelines of user activity, or any other information gathered about an individual's interaction with the rendering. Thus, the collecting mental state data involves capturing of one of a group comprising physiological data and facial data in some embodiments. Mental states may also include any type of inferred information about the individuals including, but are not limited to, frustration, confusion, disappointment, hesitation, cognitive overload, focusing, being engaged, attending, boredom, exploration, confidence, trust, delight, or satisfaction. An example of a rendering may be a checkout page on a website. If the total bill or the means of shipping is not clear, an individual may exhibit a mental state of confusion or uncertainty. In another example, a rendering may be a video trailer for a movie that will soon be released. An individual may find the plot line and action engaging, thereby exhibiting corresponding mental states such as attention and engagement which may be collected and/or inferred.

An individual may opt in 124 to the collection of mental states either before or after data is collected. In one embodiment, an individual may be asked permission to collect mental states prior to viewing or interacting with a rendering. In another embodiment, an individual may be asked permission to collect mental states after the rendering is interacted with or viewed. In this case, any information collected on mental states would be discarded if permission was not granted. In another embodiment, an individual may be asked a general question about permission for collecting of mental states prior to viewing or interacting with a rendering and then a confirmation permission requested after the rendering is interacted with or viewed. The intent of these opting in permission requests would be to give the individual control over whether mental states were collected and, further, what type of information may be used. In some embodiments however, no opt-in permission may be obtained or the opt-in may be implicit due to the circumstances of the interaction.

The mental states and rendering context may be uploaded to a server 130. The process thus may include uploading information to a server, based on the mental state data from the plurality of people who interact with the rendering. The uploading may only be for the actual data collected, and/or the uploading may be for inferred mental states. The collection of mental states 122 and capturing of rendering context 120 may be performed locally on a client computer. Alternatively, the physiological and/or facial data may be captured locally and uploaded to a server where further analysis is performed to infer the mental states. An individual may opt in 132 for allowing the uploading of information to the server. Thus the process may include opting in, by an individual from the plurality of people, to allowing uploading of mental state data to the server. The information may also include context, thus, the process may also include opting in, by an individual from the plurality of people, to allowing uploading of information to the server. In some embodiments the collected mental states may be displayed to the individual prior to uploading of information. The individual may then be asked permission to upload the information. In some embodiments an individual may be asked further permission after uploading or may be asked to confirm that the uploading which was performed is still acceptable. If permission is not granted during this opt in 132 phase, then the information would be deleted from the server and not used any further.

Mental states may be aggregated 140 between multiple individuals. A single rendering may be interacted with or viewed by numerous people. The mental states may be collected for these people and then aggregated together so that an overall reaction by the people may be determined. The aggregation may occur in the same system/process or a different system/process, than the system/process used to collect mental state or may occur on a server. The aggregated information on the mental states then may be sent between systems or between processes on the same system. Thus, the process may include receiving aggregated mental state information on the plurality of people who interact with the rendering. Individuals may opt in 142 to having their mental state information aggregated with others. In some embodiments, an individual may grant permission for their mental states to be aggregated or otherwise used in analysis. Thus the process may include opting in, by an individual from the plurality of people, to allowing information on the face to be aggregated. This information may include all facial data or may include only part of the information. For instance, some individuals may choose to have video of their faces excluded but other information on facial action units, head gestures, and the like included. In some embodiments, the aggregating is accomplished using computational aggregation. In some embodiments, analysis may be integrated over several web pages, over multiple renderings, or over a period of time. For example, a checkout experience may include four web pages and the objective is to capture the reaction to this group of four web pages. Thus the analysis may include integrating the inferred mental states for the four pages for an individual. Further, the inferred mental states for these four pages may be aggregated and thereby combined for the multiple individuals.

The flow 100 may continue with displaying the aggregated mental states with the rendering 150. Thus, the process may include displaying the aggregated mental state information with the rendering. The information associated may include facial video, other facial data, physiological data, and inferred mental states. In some embodiments, the mental states may be synchronized with the rendering using a timeline, web page sequence order, or other rendering context. The process may therefore continue with associating the aggregated mental state information with the rendering.

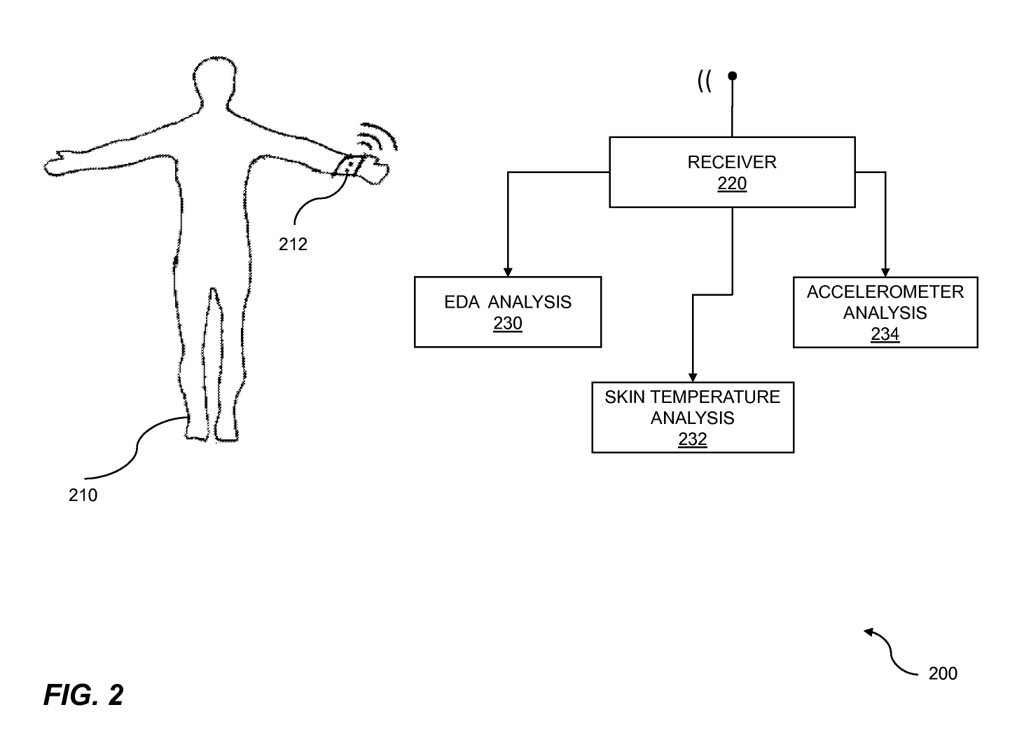

FIG. 2 is a diagram representing physiological analysis. A system 200 may analyze a person 210 for whom data is being collected. The person 210 may have a sensor 212 attached to him or her. The sensor 212 may be placed on the wrist, palm, hand, head, or other part of the body. The sensor 212 may include detectors for physiological data, such as electrodermal activity, skin temperature, accelerometer readings and the like. Other detectors for physiological data may be included as well, such as heart rate, blood pressure, EKG, EEG, further brain waves, and other physiological detectors. The sensor 212 may transmit information collected to a receiver 220 using wireless technology such as Wi-Fi, Bluetooth, 802.11, cellular, or other bands. In other embodiments, the sensor 212 may communicate with the receiver 220 by other methods such as a wired interface, or an optical interface. The receiver may provide the data to one or more components in the system 200. In some embodiments, the sensor 212 may record various physiological information in memory for later download and analysis. In some embodiments, the download of data the recorded physiological information may be accomplished through a USB port or other wired or wireless connection.

Mental states may be inferred based on physiological data, such as physiological data from the sensor, or inferred based on facial expressions and head gestures observed by a webcam. The mental states may be analyzed based on arousal and valence. Arousal can range from being highly activated, such as when someone is agitated, to being entirely passive, such as when someone is bored. Valence can range from being very positive, such as when someone is happy, to being very negative, such when someone is angry. Physiological data may include electrodermal activity (EDA) or skin conductance or galvanic skin response (GSR), accelerometer readings, skin temperature, heart rate, heart rate variability, and other types of analysis of a human being. It will be understood that both here and elsewhere in this document, physiological information can be obtained either by sensor or by facial observation. Facial data may include facial actions and head gestures used to infer mental states. Further, the data may include information on hand gestures or body language and body movements such as visible fidgets. In some embodiments these movements may be captured by cameras or by sensor readings. Facial data may include the tilting the head to the side, leaning forward, a smile, a frown, as well as many other gestures or expressions.

Electrodermal activity may be collected in some embodiments and may be collected continuously, every second, four times per second, eight times per second, 32 times per second, or on some other periodic basis. The electrodermal activity may be recorded. The recording may be to a disk, a tape, onto flash memory, into a computer system, or streamed to a server. The electrodermal activity may be analyzed 230 to indicate arousal, excitement, boredom, or other mental states based on changes in skin conductance. Skin temperature may be collected on a periodic basis and may be recorded. The skin temperature may be analyzed 232 and may indicate arousal, excitement, boredom, or other mental states based on changes in skin temperature. Accelerometer data may be collected and indicate one, two, or three dimensions of motion. The accelerometer data may be recorded. The accelerometer data may be analyzed 234 and may indicate a sleep pattern, a state of high activity, a state of lethargy, or other state based on accelerometer data. The various data collected by the sensor 212 may be used along with the facial data captured by the webcam. Thus, in some embodiments, mental state data is collected by capturing of one of a group comprising physiological data and facial data.

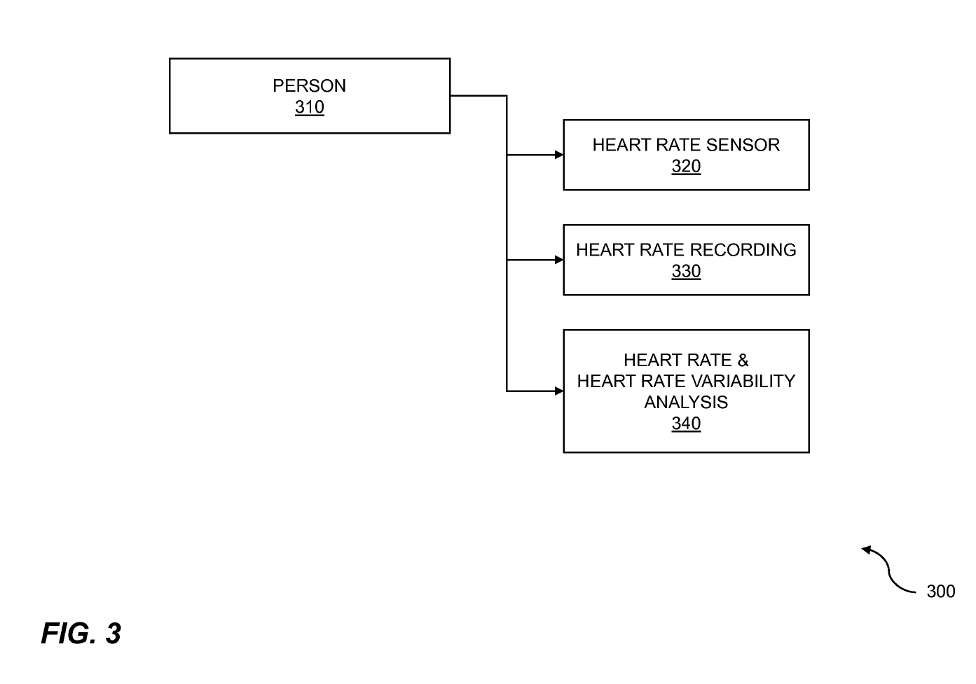

FIG. 3 is a diagram of heart related sensing. A person 310 is observed by system 300 which may include a heart rate sensor 320. The observation may be through a contact sensor or through video analysis, which enables capture of heart rate information, or other contactless sensing. In some embodiments, a webcam is used to capture the physiological data. In some embodiments, the physiological data is used to determine autonomic activity, and the autonomic activity is one of a group comprising heart rate, respiration, and heart rate variability in some embodiments. Other embodiments may determine other autonomic activity such as pupil dilation or other autonomic activities. The heart rate may be recorded 330 to a disk, a tape, into flash memory, into a computer system, or streamed to a server. The heart rate and heart rate variability may be analyzed 340. An elevated heart rate may indicate excitement, nervousness, or other mental states. A lowered heart rate may indicate calmness, boredom, or other mental states. The level of heart-rate variability may be associated with fitness, calmness, stress, and age. The heart-rate variability may be used to help infer the mental state. High heart-rate variability may indicate good health and lack of stress. Low heart-rate variability may indicate an elevated level of stress.

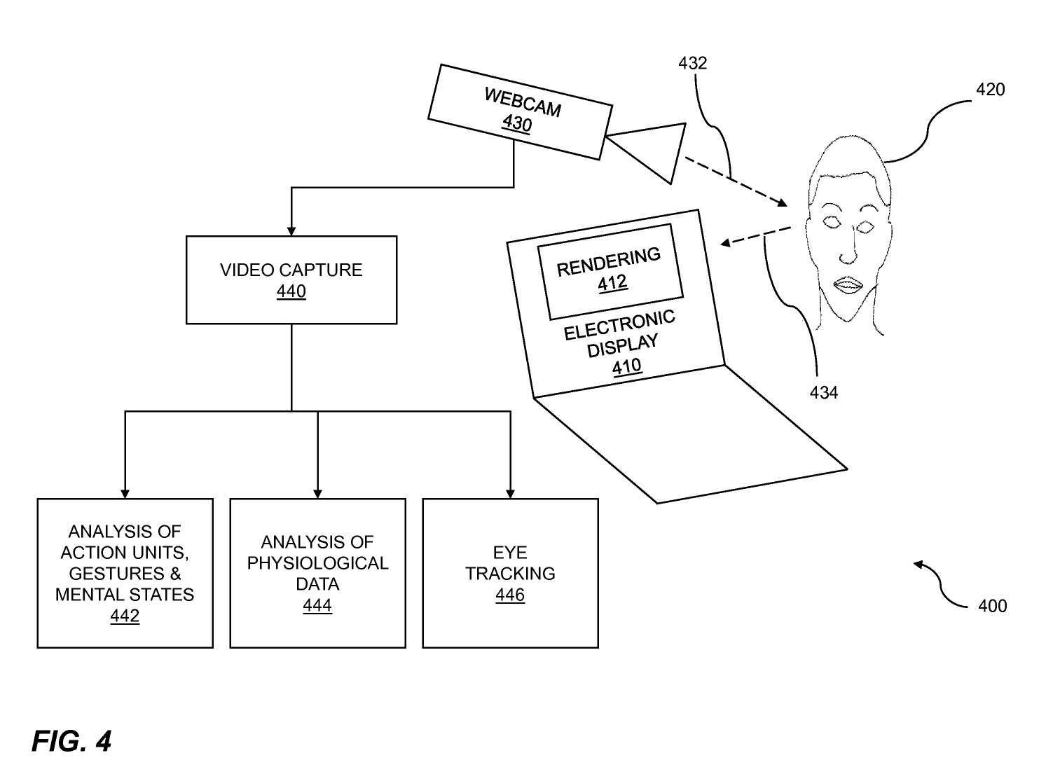

FIG. 4 is a diagram for capturing facial response to a rendering. In system 400, an electronic display 410 may show a rendering 412 to a person 420 in order to collect facial data and/or other indications of mental state. A webcam 430 a webcam is used to capture the facial data in some embodiments, although in other embodiments, a webcam 430 is used to capture one or more of the facial data and the physiological data. The facial data may include information on one or more of a group comprising facial expressions, action units, head gestures, smiles, brow furrows, squints, lowered eyebrows, raised eyebrows, and attention, in various embodiments. A webcam 430 may capture video, audio, and/or still images of the person 420. A webcam, as the term is used herein and in the claims, may be a video camera, still camera, thermal imager, CCD device, phone camera, three-dimensional camera, a depth camera, multiple webcams 430 used to show different views of the person 420 or any other type of image capture apparatus that may allow data captured to be used in an electronic system. The electronic display 410 may be a computer display, a laptop screen, a mobile device display, a cell phone display, or some other electronic display. The rendering 412 may be a landing page, a checkout page, a webpage, a website, a web-enabled application, a video on a web-enabled application, a game on a web-enabled application, or a virtual world or some other output of a web-enabled application. The rendering 412 may also be a portion of what is displayed, such as a button, an advertisement, a banner ad, a drop down menu, and a data element on a web-enabled application or other portion of the display. In some embodiments the webcam 430 may observe 432 the eyes of the person. For the purposes of this disclosure and claims, the word “eyes” may refer to either one or both eyes of an individual, or to any combination of one or both eyes of individuals in a group. The eyes may move as the rendering 412 is observed 434 by the person 420. The images of the person 420 from the webcam 430 may be captured by a video capture unit 440. In some embodiments, video may be captured while in others a series of still images may be captured. The captured video or still images may be used in one or more pieces of analysis.

Analysis of action units, gestures, and mental states 442 may be accomplished using the captured images of the person 420. The action units may be used to identify smiles, frowns, and other facial indicators of mental states. The gestures, including head gestures, may indicate interest or curiosity. For example, a head gesture of moving toward the electronic display 410 may indicate increased interest or a desire for clarification. Based on the captured images, analysis of physiological data 444 may be performed. Respiration, heart rate, heart rate variability, perspiration, temperature, and other physiological indicators of mental state can be observed by analyzing the images. So in various embodiments, a webcam is used to capture one or more of the facial data and the physiological data.

In some embodiments, a webcam is used to track the eyes. Tracking of eyes 446 to identify the rendering with which interacting is accomplished may be performed. In some embodiments, the tracking of the eyes identifies a portion of the rendering on which the eyes are focused. Thus, various embodiments may perform tracking of eyes to identify one of the rendering and a portion of the rendering, with which interacting is accomplished. In this manner, by tracking of eyes, mental states can be associated with a specific rendering or portion of the rendering. For example, if a button on a web page is unclear as to its function, a person may indicate confusion. By tracking of eyes, it will be clear that the confusion is over the button in question, rather than some other portion of the web page. Likewise, if a banner ad is present, by tracking of eyes, the portion of the banner ad which exhibits the highest arousal and positive valence may be determined. Further, in some embodiments, the process may include recording of eye dwell time on the rendering and associating information on the eye dwell time to the rendering and to the mental states. The eye dwell time can be used to augment the mental state information to indicate the level of interest in certain renderings or portion of renderings.

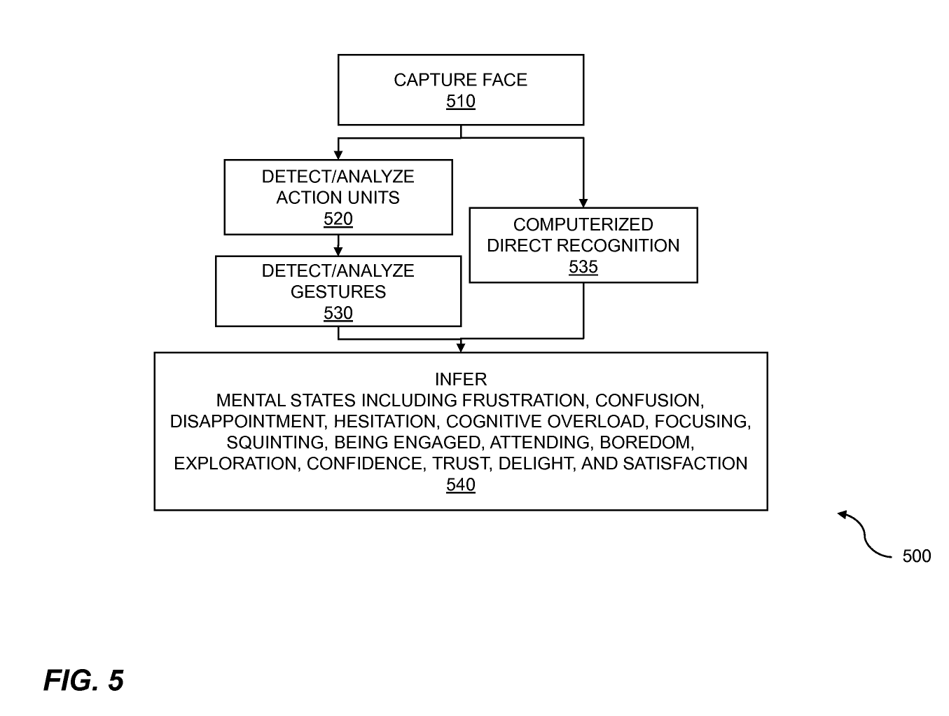

FIG. 5 is a flowchart for performing facial analysis. The flow 500 begins by capturing the face 510 of a person. The capture may be accomplished by video or by a series of still images. The flow 500 may include detection and analysis of action units 520. The action units may include the raising of an eyebrow, raising of both eyebrows, a twitch of a smile, a furrowing of the eye brows, flaring of nostrils, squinting of the eyes, and many other possibilities. These action units may be automatically detected by a computer system analyzing the video. Alternatively a combination of automatic detection by a computer system and human input may be provided to enhance the detection of the action units. The flow 500 may include detection and analysis of head and facial gestures 530. Gestures may include tilting the head to the side, leaning forward, a smile, a frown, as well as many other gestures.

In other embodiments, computerized direct recognition 535 of facial expressions and head gestures or mental states may be performed. When direct recognition is performed, feature recognition and classification may be included in the process. An analysis of mental states 540 may be performed. The mental states may include frustration, confusion, disappointment, hesitation, cognitive overload, focusing, being engaged, attending, boredom, exploration, confidence, trust, delight, and satisfaction, as well many others.

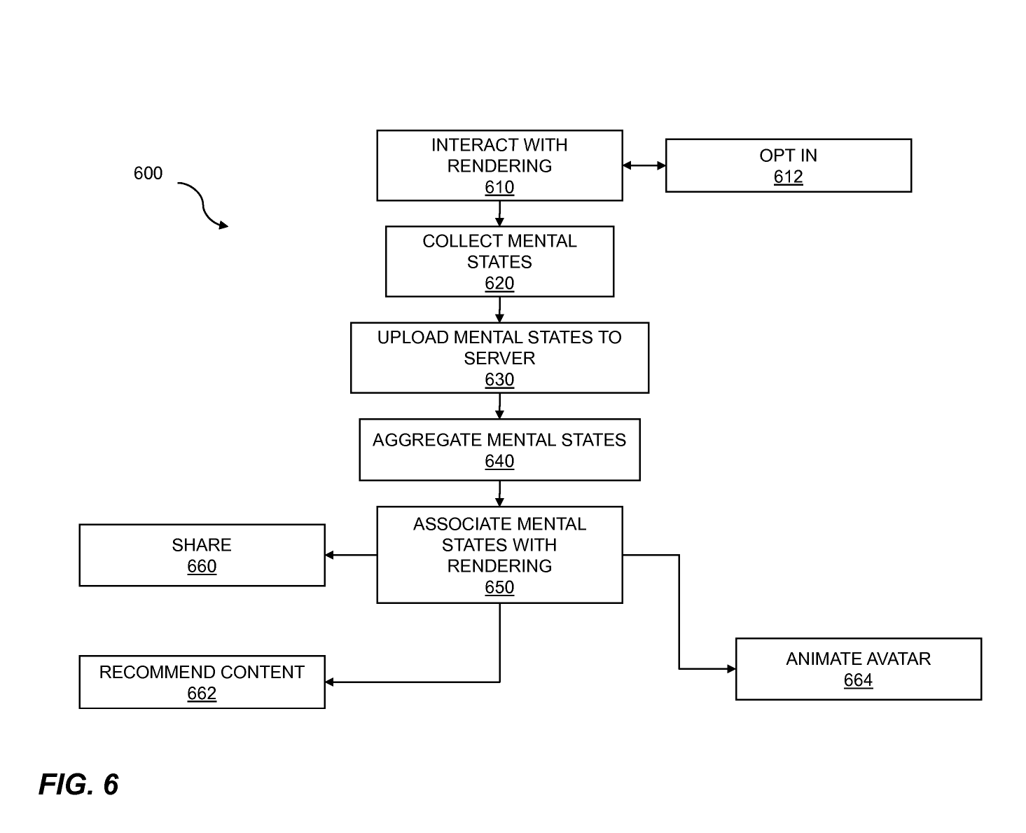

FIG. 6 is a flowchart for using mental state information. The flow 600 begins with an individual interacting with a rendering 610, such as, for example, a website. The interacting may include viewing, clicking on, or any other web enabled application oriented activity. The individual may opt in 612 to having information related to mental states collected, uploaded, aggregated, and/or shared including opting into allowing information on the face to be aggregated. The mental states may be collected 620 as the rendering is interacted with or viewed. The mental states may be inferred based on facial and physiological data which is collected. The mental states may be inferred based on computer based analysis on a client device. Some embodiments may be configured for receiving mental state data collected from a plurality of people as they interact with a rendering, as the collecting may be done on a different system. The mental states may be uploaded to a server 630. The mental states may be inferred based on computer based analysis on a server device. Further, mental state analysis may be aided by human interaction.

The mental states may be aggregated 640 with other people's mental state information which was collected. Aggregating mental state information on the plurality of people who interact with the rendering may be done in some embodiments. Receiving aggregated mental state information, based on the mental state data from the plurality of people who interact with the rendering may be done in other embodiments where the aggregating is done on a different system. Each of the people may have interacted with or viewed the same rendering. The mental states are collected and synchronized with information about the rendering. The synchronization may be based on a timeline, a sequence of web pages viewed, an eye tracking of a rendering or portion of a rendering, or some other synchronization technique. The aggregation may be by means of scaling of collected information. The aggregation may be combining of various mental states that were inferred. The aggregation may be a combination of electrodermal activity, heart rate, heart rate variability, respiration, or some other physiological reading. The aggregation may involve computational aggregation. In some embodiments, aggregation may involve noise cleaning of the data through techniques involving a low pass and/or a high pass filter or a band pass filter on the data. Normalization may occur to remove any noise spikes on the data. Noise spikes are frequently removed through nonlinear filtering such as robust statistics or morphological filters. Time shifts may occur to put the data collected on the same effective timeline. In some embodiments, this time shifting is referred to as time warping. Normalization and time warping may be interchanged in order. The data collected may be averaged. Robust statistics such as median values may be obtained. Using these techniques, outliers are removed and data below a certain threshold is discarded. Finally, visualization and display may be performed on the data. For example, electrodermal activity measurements may be aggregated using the techniques described above so that a quantitative set of numbers representing a group of people's responses may be determined. Additionally, in some embodiments, non-linear stretching may be used to focus on a small range of information. For example, a specific time range may be of particular interest due to the mental state response. Therefore the time before and after this time may be compressed while the time range of interest is expanded. In some embodiments, the aggregated mental state information may include norms derived from the plurality of people. The norms may be based on contextual information where the contextual information may be based on information from the rendering, information from sensors, or the like.

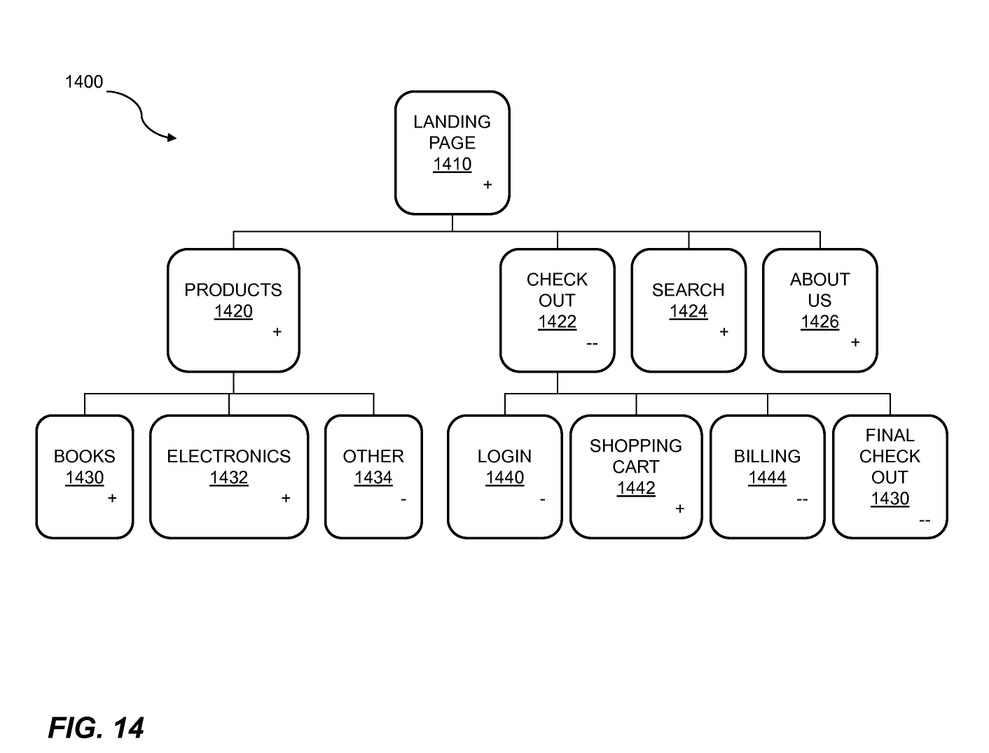

The flow 600 continues by associating the aggregated mental state information with the rendering 650. The rendering, such as a web page, video, or some other web-enabled application may have aggregated mental states associated with the rendering. In this manner, a web page button may be associated with confusion, a video trailer associated with anticipation, or a check out page or pages associated with confidence. Likewise, certain times in a video may be associated with positive mental states while other times in a video may be associated with negative mental states. FIG. 14 will provide an exemplary visual representation of mental state responses to a series of web pages.

The mental states may be shared 660. The aggregated mental state information may be shared with an individual or group of people. Mental state information from an individual may be shared with another individual or group of people. Providing the aggregated mental state information to a requester may be done in some embodiments, while displaying the aggregated mental state information with the rendering may be done in others. This sharing of information may be useful to help people see what other people liked and disliked. Similarly, content may be recommended 662. For example, a video trailer which evoked a strong arousal and a positive valence may be recommended to others who share similar mental states for other video trailers. Additionally, an avatar may be animated 664 based on the mental states. The animation may be of just a face, a head, an upper half of a person, or a whole person. The animation may be based on an individual's mental state information. Alternatively, the animation may be based on the aggregated mental state information. In embodiments, mental state information for an individual may be compared with the aggregated mental states. Differences between the individual and the aggregated mental states may be highlighted.

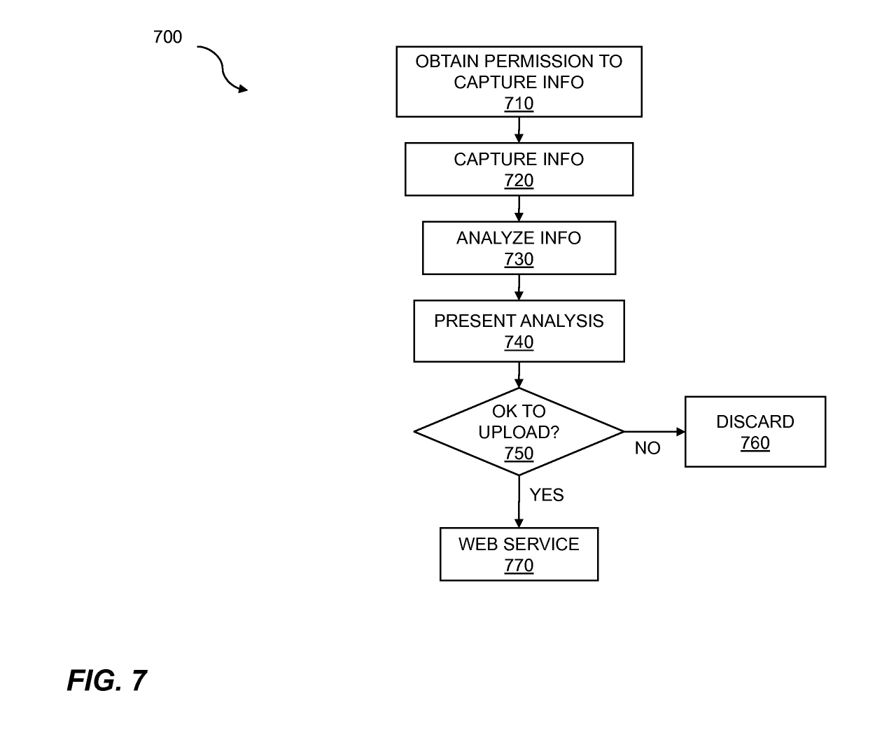

FIG. 7 is a flowchart for opting into analysis. The flow 700 may begin with obtaining permission to capture information 710 from an individual. The information being captured may include facial data, physiological data, accelerometer data, or some other data obtained in the effort to infer mental states. In some embodiments, the permission requested may be for the analysis of the information captured, such as the mental states inferred or other related results. In some embodiments, the permission may be requested at another point or points in the flow 700. Likewise, permission may be requested at each step in the collection or analysis process.

The flow 700 continues with the capture of information 720. The information captured may include facial data, physiological data, accelerometer data, or some other data. The information is analyzed 730 to infer mental states. The analysis may involve client computer analysis of facial data, head gestures, physiological data, accelerometer data, and other collected data. The results of the analysis may be presented 740 to the individual. For example, the mental states and collected information may be presented. Based on the permission requested, the client computer may determine that it is acceptable to upload 750 the captured information and/or the analysis results. A further request for permission may be requested at this time, based on the presented analysis 740. For example, allowing the opting in, by an individual from the plurality of people, to allowing uploading of mental state data. If permission is not obtained for uploading of the analysis or information, the analysis or information may be discarded 760. If the permission to upload is obtained, the information and/or analysis may be provided to a web service 770. The web service may provide additional analysis, aggregate the mental state information, or provide for sharing of the analysis or mental state information.

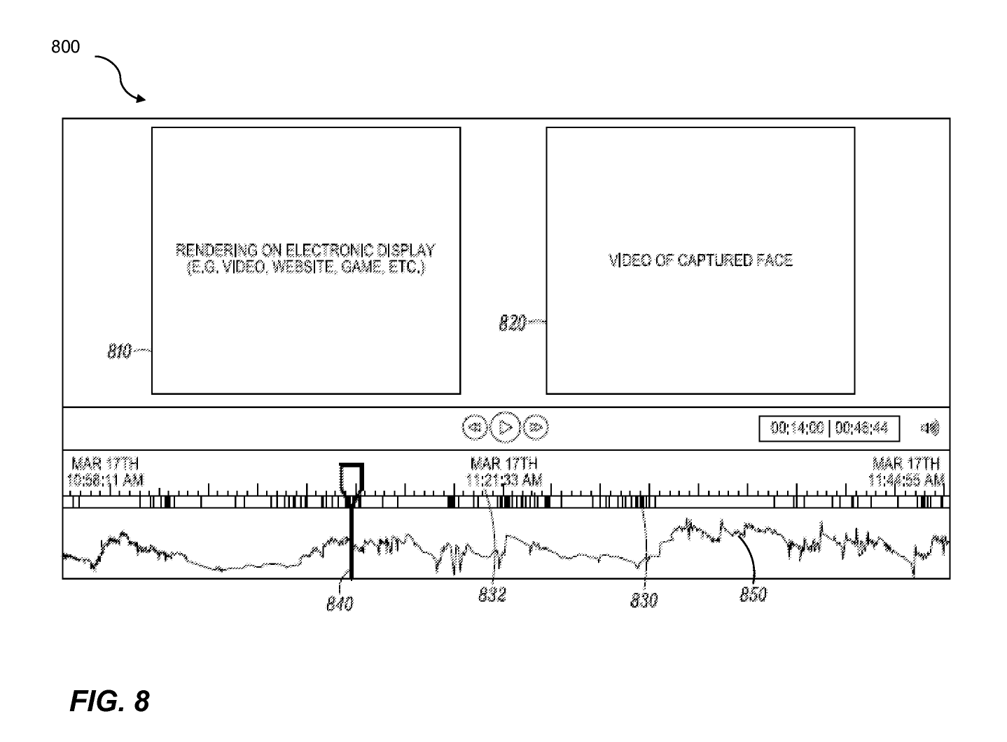

FIG. 8 is a representative diagram of a rendering and response. A display window 800 may contain the rendering 810 along with video of the person viewing the rendering 820 and may also include one or more displays of additional information. In some embodiments, each of these portions may be individual floating windows which may be repositioned as the user desires. The rendering on an electronic display 810 may be any type of rendering, including any rendering described herein, such as, without limitation, a landing page, a checkout page, a webpage, a website, a web-enabled application, a video on a web-enabled application, a game on a web-enabled application, or a virtual world. This rendering display 810 shows the user of display window 800 the same rendering as the individual on whom mental state information was captured. In one example, the rendering may be a video and the video may play out in synch with the video of the captured face 820 for the individual. In some embodiments, the rendering 810 may indicate where eyes are tracking For instance if the eyes of the individual viewing the rendering have been tracked to a particular button on a webpage, then the button may be highlighted. Alternatively, a box or oval may be shown on the rendering 810 which indicates the portion of the screen on which the person's eyes were focused. In this manner, the eye tracking will indicate the focus of the person while the remainder of the window 800 may display mental state information about the person's reaction to the area of that focus.

Various information and analysis results may also be shown. In some embodiments, the additional information may be shown in the display window 800 below the rendering 810 and the video 820. Any type of information may be shown, including mental state information from an individual, aggregated mental state information from a group of people, or other information about the rendering 810, the video 820, the individual or group of people from whom the mental state information was captured, or any other type of information. Thus, creating a visual representation of one or more of the aggregated mental state information and mental state information on an individual from the plurality of people may be done in some embodiments. The mental state information may include any type of mental state information described herein, including electrodermal activity, accelerometer readings, frown markers, smile markers, as well as numerous other possible physiological and mental state indicators. By way of example, in window 800, a smile marker track 830 is provided. Where a narrow line on the smile marker track 830 exists, a hint of smile was detected. Where a solid dark line is shown, a broad smile was detected that lasted a while. This smile marker track may have a timeline 832 as shown, and the timeline 832 may also have a slider bar 840 shown. The slider bar may be moved to various points on the timeline 830 and the rendering 810 and video 820 may each show what occurred at that point in time. By further example, an electrodermal activity track 850 is shown as well. While window 800 may show an individual, this window or set of windows may create a visual representation of the aggregated mental state information as well. For instance, once electrodermal activity information has been aggregated for a group of people, the aggregated electrodermal activity may be displayed for the rendering 810. As stated earlier, numerous displays of information and analysis are possible in this window or set of windows. These displays can be for the individual or for an aggregated group of people.

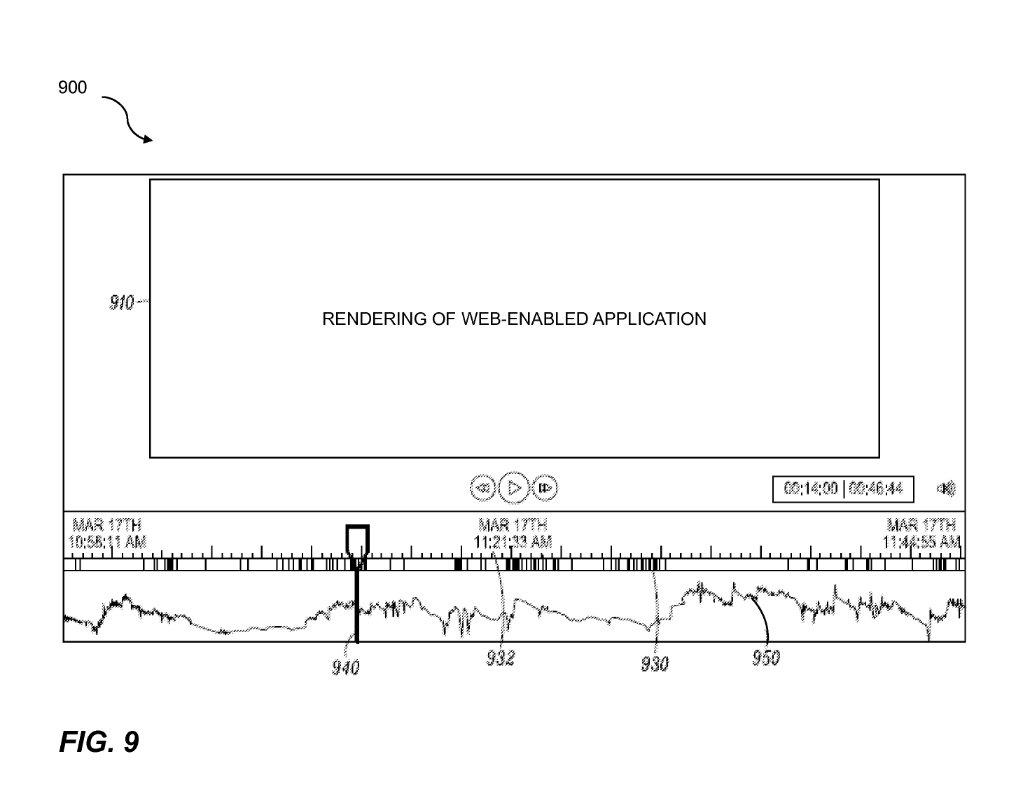

FIG. 9 is a representative diagram of a rendering and an aggregated response. A display window 900 may contain the rendering 910 of a web-enabled application. This rendering may be that which was shown on an electronic display to multiple people. The rendering on an electronic display 910 may be any type of rendering, including any rendering described herein, such as, without limitation, a landing page, a checkout page, a webpage, a website, a mobile-device application, a cell-phone application, a web-enabled application, a video on a web-enabled application, a game on a web-enabled application, or a virtual world. This rendering 910 may show the user of display window 900 the same rendering as the multiple people on whom mental state information was captured. In some embodiments, the rendering 910 may indicate where a majority of eyes from the multiple people were tracking. For instance a button may be highlighted or a box or oval may be shown on the rendering 910 which indicates the portion of the screen on which the majority of people's eyes were focused.

Various information and aggregated analysis results may be shown including, for example, electrodermal activity, accelerometer readings, frown markers, smile markers, as well as numerous other possible physiological and mental state indicators. By way of example, in window 900, a smile marker track 930 is provided. Where a narrow line on the smile marker track 930 exists, a hint of smile was detected as a majority response of the multiple people. Where a solid dark line is shown, a broad smile that lasts may have been detected as a majority response of multiple people. This smile marker track may have a timeline 932 as shown and the timeline 932 may also have a slider bar 940 shown. The slider bar may be moved to various points on the timeline 930 and the rendering 910 may show what occurred at that point in time, synchronizing the aggregated mental state information with the rendering. By further example, an aggregated electrodermal activity track 950 may also be included. As stated earlier, numerous displays of information and analysis are possible in this window or set of windows. In some embodiments, each of these portions may be individual floating windows which may be repositioned as the user desires.

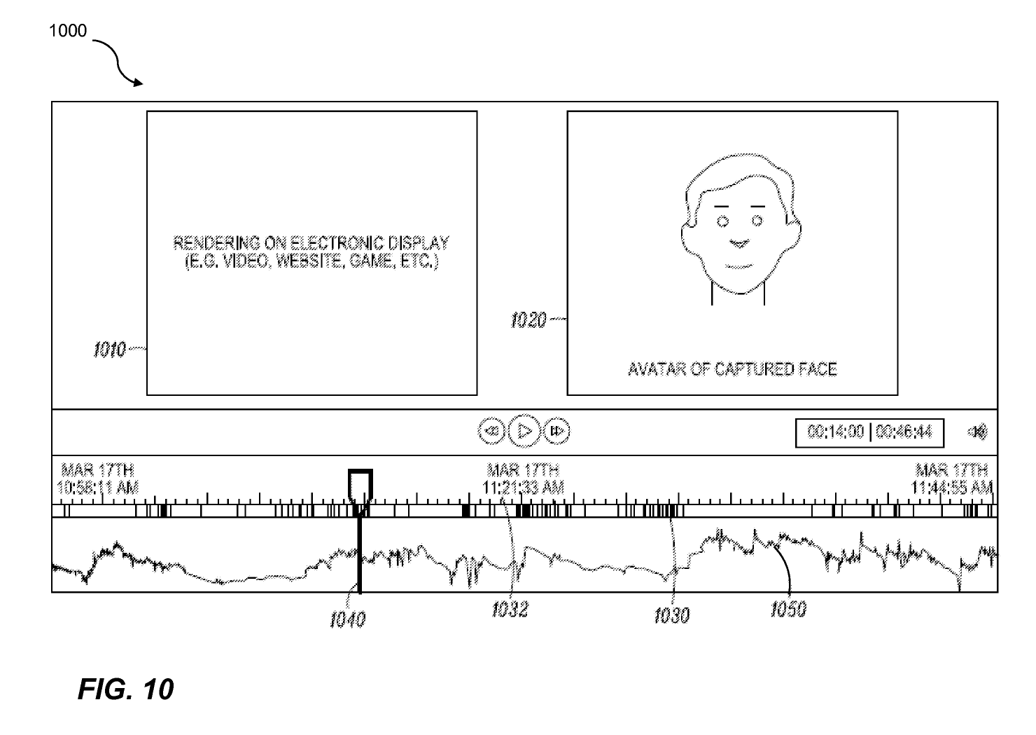

FIG. 10 is a representative diagram of a rendering and response with avatar. A window 1000 may be shown which includes, for example, display of a rendering 1010, an avatar of the captured face 1020, a smile track 1030, a timeline 1032, a slide bar 1040, and an electrodermal activity track 1050. Numerous other displays of information are possible as well. Each of the elements mentioned may be shown in window 1000 or may be shown in another floating window. The avatar 1020 represents the person who viewed the rendering without showing video of the person. By using an avatar, a person's identity may be removed but indications of smiling, frowning, laughing, and other facial expressions may still be shown by means of the avatar. The avatar may show just a face, a whole head, an upper body, or a whole person. The avatar may, in some embodiments, reflect the characteristics of the individual that it represents, including gender, race, hair color, eye color, and various other aspects of the individual. In other embodiments, the concepts may include animating an avatar to represent the aggregated mental state information. An avatar may then describe a group's response to the rendering 1010. For example, if the majority of people were engaged and happy then the avatar might be shown with a smile and with a head that is tilted forward. So as described above, the concept in some embodiments may include animating an avatar to represent one or more of the aggregated mental state information and mental state information on an individual from the plurality of people.

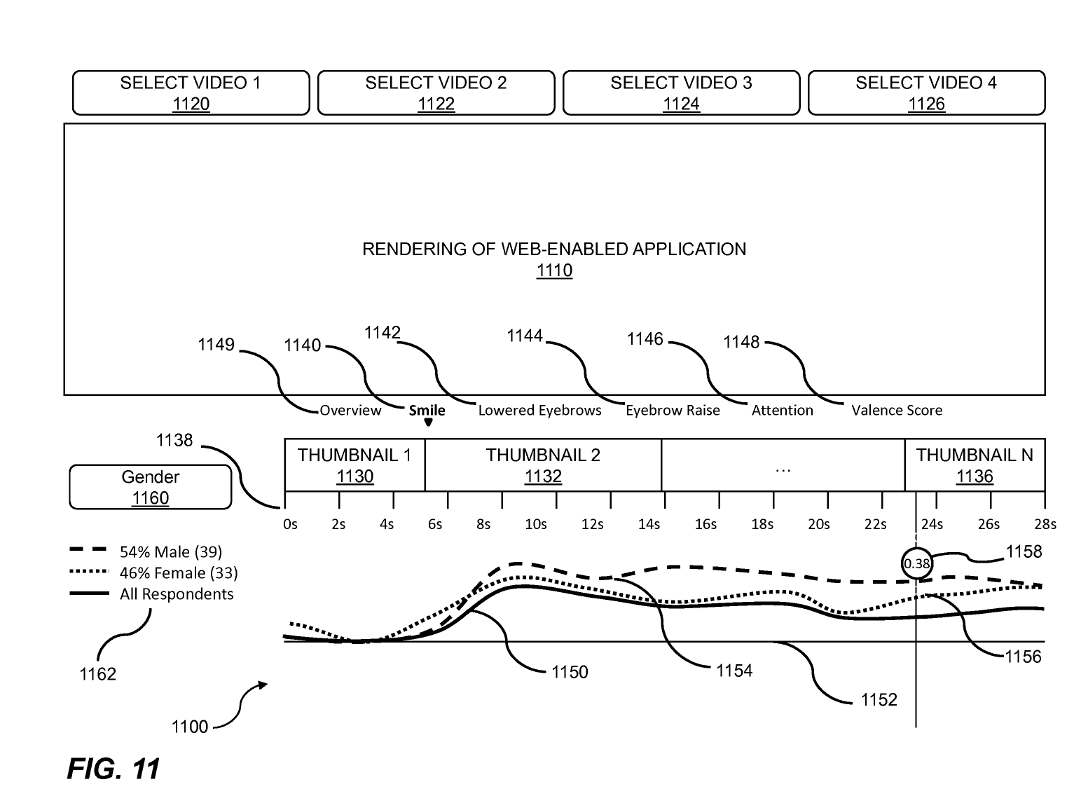

FIG. 11 is a graphical representation of mental state analysis. A window 1100 may be shown which includes, for example, rendering of the web-enabled application 1110 having associated mental state information. The rendering in the example shown is a video but may be any other sort of rendering in other embodiments. A user may be able to select between a plurality of renderings using various buttons and/or tabs such as Select Video 1 button 1120, Select Video 2 button 1122, Select Video 3 button 1124, and Select Video 4 button 1126. Various embodiments may have any number of selections available for the user and some may be other types of renderings instead of video. A set of thumbnail images for the selected rendering, that in the example shown include thumbnail 1 1130, thumbnail 2 1132, through thumbnail N 1136 may be shown below the rendering along with a timeline 1138. Some embodiments may not include thumbnails, or have a single thumbnail associated with the rendering, and various embodiments may have thumbnails of equal length while others may have thumbnails of differing lengths. In some embodiments, the start and/or end of the thumbnails may be determined by the editing cuts of the video of the rendering while other embodiments may determine a start and/or end of the thumbnails based on changes in the captured mental states associated with the rendering.

Some embodiments may include the ability for a user to select a particular type of mental state information for display using various buttons or other selection methods. In the example shown, the smile mental state information is shown as the user may have previously selected the Smile button 1140. Other types of mental state information that may be available for user selection in various embodiments may include the Lowered Eyebrows button 1142, Eyebrow Raise button 1144, Attention button 1146, Valence Score button 1148 or other types of mental state information, depending on the embodiment. An Overview button 1149 may be available to allow a user to show graphs of the multiple types of mental state information simultaneously.

Because the Smile option 1140 has been selected in the example shown, smile graph 1150 may be shown against a baseline 1152 showing the aggregated smile mental state information of the plurality of individuals from whom mental state data was collected for the rendering 1110. Male smile graph 1154 and female smile graph 1156 may be shown so that the visual representation displays the aggregated mental state information on a demographic basis. The various demographic based graphs may be indicated using various line types as shown or may be indicated using color or other method of differentiation. A slider 1158 may allow a user to select a particular time of the timeline and show the value of the chosen mental state for that particular time. The slider may show the same line type or color as the demographic group whose value is shown.

Various types of demographic based mental state information may be selected using the demographic button 1160 in some embodiments. Such demographics may include gender, age, race, income level, or any other type of demographic including dividing the respondents into those respondents that had a higher reaction from those with lower reactions. A graph legend 1162 may be displayed indicating the various demographic groups, the line type or color for each group, the percentage of total respondents and or absolute number of respondents for each group, and/or other information about the demographic groups. The mental state information may be aggregated according to the demographic type selected. Thus, aggregation of the aggregated mental state information is performed on a demographic basis so that mental state information is grouped based on the demographic basis for some embodiments.

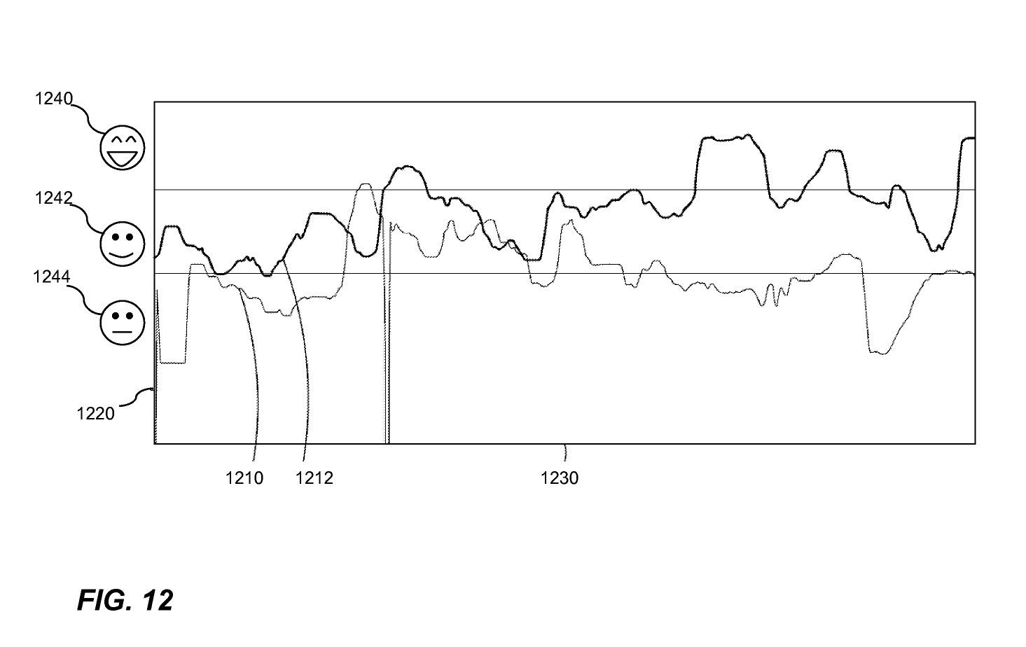

FIG. 12 is a graphical representation of mental state analysis along with an aggregated result from a group of people. This rendering may be displayed on a web page, web enabled application, or other type of electronic display representation. A graph 1210 may be shown for an individual on whom affect data is collected. Another graph 1212 may be shown for affect collected on another individual or aggregated affect from multiple people. The mental state analysis may be based on facial image or physiological data collection. In some embodiments, the graph 1210 may indicate the amount or probability of a smile being observed for the individual. A higher value or point on the graph may indicate a stronger or larger smile. In certain spots the graph may drop out or degrade when image collection lost or was not able to identify the face of the person. The probability or intensity of an affect may be given along the y-axis 1220. A timeline may be given along the x-axis 1230. The aggregated information may be based on taking the average, median, or other statistical or calculated value based on the information collected from a group of people. In some embodiments, aggregation of the aggregated mental state information is accomplished using computational aggregation.

In some embodiments, graphical smiley face icons 1240, 1242, and 1244 may be shown providing an indication of the amount of a smile or other facial expression. A first very broad smiley face icon 1240 may indicate a very large smile being observed. A second normal smiley face icon 1242 may indicate a smile being observed. A third face icon 1240 may indicate no smile. Each of the icons may correspond to a region on the y-axis 1220 that indicate the probability or intensity of a smile.

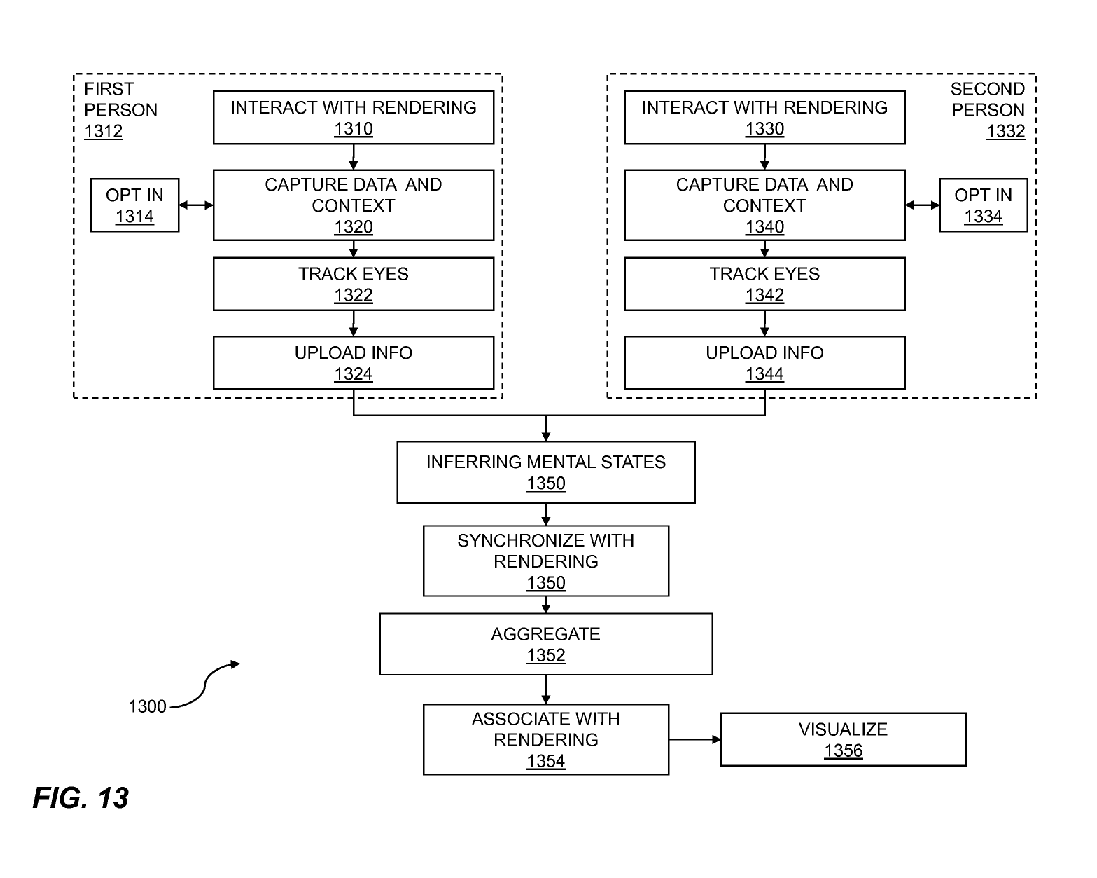

FIG. 13 is a flowchart for analyzing affect from rendering interaction. The flow 1300 for analyzing renderings on electronic displays begins with interacting with a rendering 1310 on an electronic display by a first person 1312. The rendering may be any type of rendering including those renderings described herein. In some embodiments, there may be a query for the first person 1312 to opt in 1314 to the process of capturing data. If allowable by the first person, the flow 1300 may continue by capturing context and capturing data 1320 on the first person into a computer system as the first person interacts with the rendering on the electronic display. In some embodiments, capturing data involves capture of one of a group comprising physiological data and facial data. The captured data may include electrodermal, accelerometer, and/or other data. The captured context may be a timeline, a sequence of web pages, or some other indicator of what is occurring in the web enabled application.

The eyes may be tracked 1322 to determine where the first person 1312 is focused on the display. The flow 1300 includes uploading information 1324 to a server on the data which was captured on the first person. Permission may again be asked before the upload of information.

The flow 1300 continues with interacting with the rendering 1330 by a second person 1332. In some embodiments, there may be a query for the second person 1332 to opt in 1334 to the process of capturing data. If allowable by the second person, the flow 1300 may continue by capturing context and capturing data 1340 on the second person as the second person interacts with the rendering. The eyes may be tracked 1342 to determine where the second person 1332 is focused on the display. The flow 1300 may include uploading information 1344 to the server on the data which was captured on the second person. Permission may again be asked before the upload of information.

The flow 1300 continues with the inferring of mental states for the first person who interacted with the rendering based on the data which was captured for the first person and inferring of mental states for the second person who interacted with the rendering based on the data which was captured for the second person 1350. This inferring may be done on the client computer of the first and second person. Alternatively, the inferring of mental states may be performed on the server computer after the upload of information or on some other computer with access to the uploaded information. The inferring of mental states is based on one of a group comprising physiological data and facial data, in some embodiments, and may include inferring of mental states based on the mental state data collected from the plurality of people. The mental states may be synchronized with the rendering 1350. In one embodiment, this synchronization may correlate the mental states with a timeline that is part of a video. In embodiments, the synchronization may correlate the mental states with a specific web page or a certain sequence of web pages. The synchronization may be performed on the first and second person's client computer or may be performed on a server computer after uploading or by some other computer.

The flow 1300 continues with aggregating 1352 information on the mental states of the first person with the mental states of the second person resulting in aggregated mental state information. The aggregating may include computational aggregation. The aggregation may include combining electrodermal activity or other readings from multiple people. The flow 1300 continues with associating the aggregated mental state information to the rendering 1354 with which the first person and the second person interacted. The associating of the aggregated mental state information allows recall and further analysis of the rendering and people's mental state reactions to the rendering. The flow 1300 continues with visualization 1356 of the aggregated mental state information. This visualization may include graphical or textual presentation. The visualization may also include a presentation in the form of an avatar. The flow 1300 may continue with any number of people's data being captured, mental states being inferred, and all other steps in the flow.

FIG. 14 is an example embodiment of a visual representation of mental states, in this example, a series of web pages 1400 with which there has been interaction. These web pages include a landing page 1410, a products page 1420, a check-out page 1422, a search page 1424, and an about us page 1426. Some of these pages may in turn have sub pages, such as the products page 1420, which has sub pages of a books page 1430, an electronics page 1432, and other product pages represented by the other page 1434. In some embodiments one or more of these pages may have further sub pages. Further, the check-out page 1422 has sub pages of a login page 1440, a shopping cart page 1442, a billing page 1444, and a final check out page 1430. As an individual interacts with these pages mental states may be inferred. Further, as multiple people interact with these pages, aggregated information on inferred mental states may be accumulated. Detailed results may be accumulated on each of these pages. These detailed results may be presented. Alternatively, simplified analysis may be presented that gives positive, slightly negative, and negative indications. In some embodiments very positive or neutral responses may also be shown. In the series of web pages 1400, a positive impression is shown as a “+” in the lower right hand corner of the web page box, such as the landing page 1410. A “+” may denote a positive mental state for an individual or aggregated group of people. A slightly negative response may be denoted by a “−” in the bottom right of the web page box, such as the login page 1440. A “−” may indicate confusion. A very negative reaction may be indicated by a “− −” in the lower right corner of the web page box such as the billing page 1444. A “− −” may denote anger, frustration, or disappointment. In some embodiments, colors may be used to represent the positive, slightly negative, and very negative reactions. Such colors may be green, yellow, and red, respectively. Any of the methods described, or other methods of displaying the aggregated mental state information may be used for creating a visual representation of the aggregated mental state information.

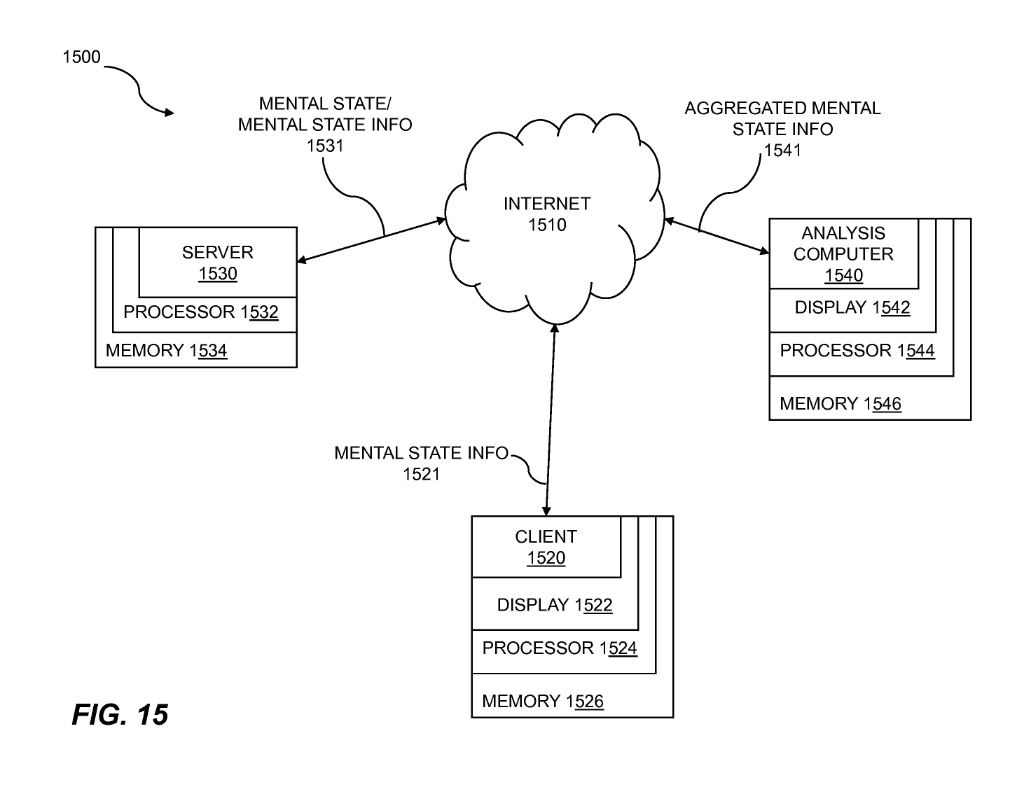

FIG. 15 is a diagram of a system 1500 for analyzing web-enabled application traffic states utilizing multiple computers. The internet 1510, intranet, or other computer network may be used for communication between the various computers. A client computer 1520 has a memory 1526 which stores instructions and one or more processors 1524 attached to the memory 1526 wherein the one or more processors 1524 can execute instructions. The client computer 1520 also may have an internet connection to carry mental state information 1521 and a display 1522 that may present various renderings to a user. The client computer 1520 may be able to collect mental state data from a plurality of people as they interact with a rendering. In some embodiments there may be multiple client computers 1520 that each may collect mental state data from one person or a plurality of people as they interact with a rendering. In other embodiments, the client computer 1520 may receive mental state data collected from a plurality of people as they interact with a rendering. Once the mental state data has been collected the client computer may upload information to a server 1530, based on the mental state data from the plurality of people who interact with the rendering. The client computer 1520 may communicate with the server 1530 over the internet 1510, some other computer network, or by other method suitable for communication between two computers. In some embodiments, the server 1530 functionality may be embodied in the client computer.

The server 1530 may have an internet connection for mental states or mental state information 1531 and have a memory 1534 which stores instructions and one or more processors 1532 attached to the memory 1534 wherein the one or more processors 1532 can execute instructions. The server 1530 may receive mental state information collected from a plurality of people as they interact with a rendering from the client computer 1520 or computers, and may aggregate mental state information on the plurality of people who interact with the rendering. The server 1530 may also associate the aggregated mental state information with the rendering and also with the collection of norms for the context being measured. In some embodiments the server 1530 may also allow user to view and evaluate the mental state information that is associated with the rendering, but in other embodiments, an analysis computer 1540 may request the aggregated mental state information 1541 from the server 1530. The server 1530 may then provide the aggregated mental state information 1541 to a requester, the analysis computer 1540. In some embodiments the client computer 1520 may also function as the analysis computer 1540.

The analysis computer 1540 may have a memory 1546 which stores instructions and one or more processors 1544 attached to the memory 1546 wherein the one or more processors 1544 can execute instructions. The analysis computer may use its internet, or other computer communication method, to request the aggregated mental state information 1541 from the server. The analysis computer 1540 may receive aggregated mental state information 1541, based on the mental state data from the plurality of people who interact with the rendering and may present the aggregated mental state information with the rendering on a display 1542. In some embodiments, the analysis computer may be set up for receiving mental state data collected from a plurality of people as they interact with a rendering, in a real-time or near real-time embodiment. In at least one embodiment, a single computer may incorporate the client, server and analysis functionality.

Each of the above methods may be executed on one or more processors on one or more computer systems. Embodiments may include various forms of distributed computing, client/server computing, and cloud based computing. Further, it will be understood that for each flow chart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, and so on. Any and all of which may be generally referred to herein as a “circuit,” “module,” or “system.”

A programmable apparatus which executes any of the above mentioned computer program products or computer implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM, Flash, MRAM, FeRAM, or phase change memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

1. A computer implemented method for analyzing web-enabled application traffic comprising:
collecting mental state data from a plurality of people as they interact with a rendering;
uploading information, to a server, based on the mental state data from the plurality of people who interact with the rendering;
receiving aggregated mental state information on the plurality of people who interact with the rendering; and
displaying the aggregated mental state information with the rendering.

2. The method of claim 1 wherein the aggregated mental state information includes norms derived from the plurality of people.

3. The method of claim 2 wherein the norms are based on contextual information.

4. The method of claim 1 further comprising associating the aggregated mental state information with the rendering.

5. The method according to claim 1 wherein the rendering is one of a group comprising a button, an advertisement, a banner ad, a drop down menu, and a data element on a web-enabled application.

6. The method according to claim 1 wherein the rendering is one of a group comprising a landing page, a checkout page, a webpage, a website, a web-enabled application, a video on a web-enabled application, a game on a web-enabled application, and a virtual world.

7. The method according to claim 1 wherein the collecting mental state data involves capturing of one of a group comprising physiological data and facial data.

8. The method according to claim 7 wherein a webcam is used to capture one or more of the facial data and the physiological data.

9. The method according to claim 8 wherein the physiological data is used to determine autonomic activity.

10. The method according to claim 9 wherein the autonomic activity is one of a group comprising heart rate, respiration, and heart rate variability.

11. The method according to claim 7 wherein the facial data includes information on one or more of a group comprising facial expressions, action units, head gestures, smiles, brow furrows, squints, lowered eyebrows, raised eyebrows, and attention.

12. The method according to claim 1 further comprising tracking of eyes to identify the rendering with which interacting is accomplished.

13. The method according to claim 12 wherein the tracking of the eyes identifies a portion of the rendering on which the eyes are focused.

14. (canceled)

15. The method according to claim 12 further comprising recording of eye dwell time on the rendering and associating information on the eye dwell time to the rendering and to the mental state data.

16. (canceled)

17. The method according to claim 1 further comprising opting in, by an individual from the plurality of people, to allowing facial information to be aggregated.

18-19. (canceled)

20. The method according to claim 1 wherein aggregation of the aggregated mental state information is performed on a demographic basis so that mental state information is grouped based on the demographic basis.

21. The method according to claim 1 further comprising creating a visual representation of one or more of the aggregated mental state information and mental state information on an individual from the plurality of people.

22. The method according to claim 21 wherein the visual representation displays the aggregated mental state information on a demographic basis.

23. (canceled)

24. The method according to claim 1 further comprising synchronizing the aggregated mental state information with the rendering.

25. The method according to claim 1 further comprising capturing contextual information about the rendering.

26. The method according to claim 25 wherein the contextual information includes one or more of a timeline, a progression of web pages, and an actigraph.

27. The method of claim 1 further comprising inferring of mental states based on the mental state data collected from the plurality of people.

28. The method according to claim 27 wherein the mental states include one of a group comprising frustration, confusion, disappointment, hesitation, cognitive overload, focusing, being engaged, attending, boredom, exploration, confidence, trust, delight, and satisfaction.

29. A computer program product embodied in a non-transitory computer readable medium for analyzing web-enabled application traffic, the computer program product comprising:
code for collecting mental state data from a plurality of people as they interact with a rendering;
code for uploading information, to a server, based on the mental state data from the plurality of people who interact with the rendering;
code for receiving aggregated mental state information on the plurality of people who interact with the rendering; and
code for displaying the aggregated mental state information with the rendering.

30. A system for analyzing web-enabled application traffic states comprising:
a memory which stores instructions;
one or more processors attached to the memory wherein the one or more processors, when executing the instructions which are stored, are configured to:
collect mental state data from a plurality of people as they interact with a rendering;
upload information, to a server, based on the mental state data from the plurality of people who interact with the rendering;
receive aggregated mental state information on the plurality of people who interact with the rendering; and
display the aggregated mental state information with the rendering.

31-33. (canceled)
